(19) United States
(12) Patent Application Publication
(10) Pub. No.: US 2016/0291024 A1
(43) Pub. Date: Oct. 6, 2016

(54) Biomarkers for Ovarian Cancer

(71) Applicant: THE BOARD OF TRUSTEES OF THE LELAND STANFORD JUNIOR UNIVERSITY, Stanford, CA (US)

(72) Inventors: Atul J. Butte, Menlo Park, CA (US); Linda Anne Szabo, Belmont, CA (US); Purvesh Khatri, Menlo Park, CA (US); Bruce Xuefeng Ling, Palo Alto, CA (US)

(73) Assignee: THE BOARD OF TRUSTEES OF THE LELAND STANFORD JUNIOR UNIVERSITY, Stanford, CA (US)

(21) Appl. No.: 14/914,245

(22) PCT Filed: Sep. 17, 2014
(86) PCT No.: PCT/US14/56031

§ 371 Date: Feb. 24, 2016

Related U.S. Application Data
(60) Provisional application No. 61/879,051, filed on Sep. 17, 2013.

(51) Int. Cl. G01N 33/574

(52) U.S. Cl. CPC G01N 33/57449; G01N 2333/91205; G01N 2333/914; C12Y 207/11001; C12Y 306/04

(57) ABSTRACT

Biomarkers and biomarker panels are provided for making ovarian cancer assessments, for example, diagnosing an ovarian cancer, predicting responsiveness of an ovarian cancer to an ovarian cancer therapy, and monitoring an ovarian cancer. A patient may further be treated in accordance with the classification. Also provided are methods, reagents, devices and kits for the use of these biomarkers in making ovarian cancer assessments.

### FIELD OF THE INVENTION

This invention pertains to biomarkers for use in making ovarian cancer assessments.

### BACKGROUND OF THE INVENTION

Ovarian cancer is the leading cause of gynecologic cancer death and the fifth leading cause of cancer death in North American women, primarily due to lack of early symptoms or effective screening. Early detection of ovarian cancer increases the 5-year survival rate from less than 30% to 70%-90%, but less than 20% of cases are diagnosed early. Results from the Prostate, Lung, Colorectal, and Ovarian Cancer Screening Trial (PLCO) provided conclusive evidence that the current standard of care, a blood test for the CA-125 protein and trans-vaginal ultrasound, does not improve ovarian cancer early detection rates or survival. Several new early detection markers have been proposed in recent years, but to date all have failed validation. The present invention addresses these issues.

### SUMMARY OF THE INVENTION

Biomarkers and biomarker panels are provided for making ovarian cancer assessments, for example, diagnosing an ovarian cancer, predicting responsiveness of an ovarian cancer to an ovarian cancer therapy, and monitoring an ovarian cancer. A report may be provided to the patient of the assessment. Also provided are methods, reagents, devices and kits for the use of these biomarkers in making ovarian cancer assessments. Patients can further be treated with in accordance with the assessment of responsiveness.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1 depicts the method used to identify the 160 genes of the human genome that are most relevant to ovarian cancer. A. 7 datasets containing gene expression of both high grade serous ovarian cancer and controls were downloaded from The Gene Expression Omnibus. B. A forest plot representative of the output from the meta-analysis in (A) for a single gene (gene not specified in this case as it is just an overview of the method). The y axis lists the study IDs, while the x axis plots the Log2-fold change in expression (0 means no difference between cases and controls in that study, 1 means twice as much expression in cases compared to controls, etc.) The size of the blue square is proportional to the number of patients in the study and is centered at the mean log fold change for that study. The horizontal lines show the 95% confidence interval of this estimate for this single study. Then the summary statistic is shown by the yellow diamond. This is a weighted average of all the individual studies (so larger studies with smaller confidence intervals have more of an effect on that final summary statistic). The width of the diamond indicates the 95% confidence interval for that summary statistic. Each gene measured is represented by such a plot, and the genes are ranked based on the summary statistic (also called the effect size) and the statistical significance. Based on the filters applied for considering something significantly overexpressed (log fold change>0.75, p-value<0.001, FDR<0.001), 160 genes were identified that are overexpressed across all 7 studies reviewed.

FIG. 2 demonstrates that in an independent cohort, top candidates identified by the methodology described in FIG. 1 distinguish cancer cases and controls better than an art-recognized panel of biomarkers. A. The top candidate genes were validated using The Cancer Genome Atlas (TCGA) microarray data from 591 ovarian cancer cases and 8 controls. Combined expression of 4 genes distinguishes the cancer from control groups. B. For comparison, OvaSure is a blood-based biomarker panel marketed briefly in 2008 for early detection of ovarian cancer. OvaSure contains 4 proteins elevated in the serum of cancer cases. Each green dot is a patient, and the position along the y axis indicates that person's combined score based on all 4 genes. A geometric mean of the values of the 4 genes was used as output from the microarray experiment (the 4 values are multiplied together and then the 4^{th} root is taken).

FIG. 3 demonstrates how top-ranked biomarkers for early detection clearly distinguish early stage ovarian cancer cases (stage I or stage II) from normal individuals. The initial list of candidate biomarkers was prioritized based on how well the biomarkers separate normal controls from early stage cancer and how well they correlated with patient survival in the TCGA cohort. The optimal panel consists of the top 5 biomarkers on the list. Comparable results were obtained with late stage ovarian cancer cases.

FIG. 4 demonstrates that early detection biomarker candidates outperform OvaSure in a separate cohort. The early detection biomarker panel was validated in a second independent cohort (GSE4122) consisting of 32 ovarian serous adenocarcinoma cases and 32 normal or benign controls. The 3 candidate biomarkers measured in this cohort achieved a higher AUC than the 3 OvaSure genes.

FIG. 5 shows the results of a pilot validation study of two novel early detection biomarkers in human plasma. After filtering the list of candidate biomarkers from meta-analysis against proteome databases, 2 proteins (PRKDC and RAD45L) were selected for a pilot validation study in the plasma of 12 pre-treatment ovarian cancer patients and 12 age-, gender-, and ethnicity-matched controls. 4 of the 6 biomarkers in the discontinued OvaSure panel (CA125, OPN, LEP, ILGF2) were tested as positive controls. Each dot represents the ELISA value for a single patient. Blue arrow marks the result of the stage I sample; red arrow marks the result of the stage II sample. The remaining samples are stage III or IV. In each panel, the plot on the right shows values for cancer cases and the plot on the left shows values for age-, race-, and gender-matched controls.

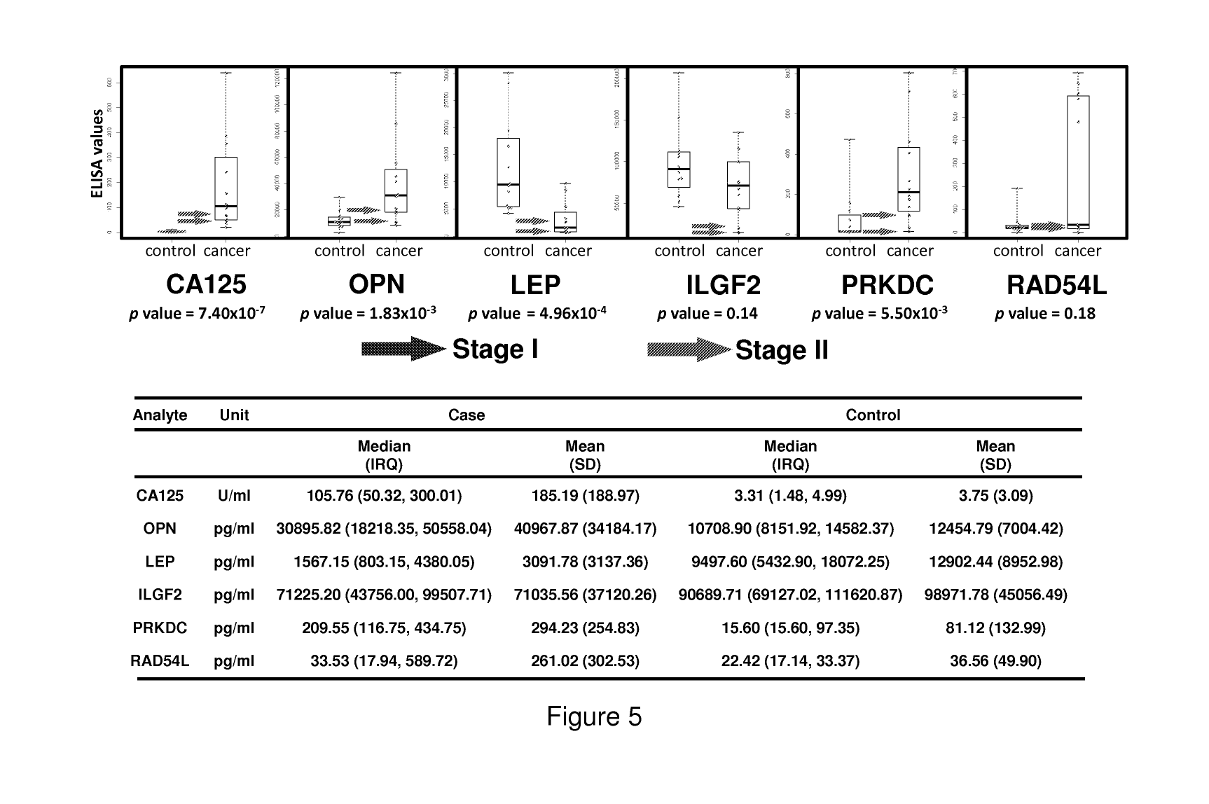

### DETAILED DESCRIPTION OF THE INVENTION

Biomarkers are provided for making ovarian cancer assessments, for example, diagnosing an ovarian cancer, predicting responsiveness of an ovarian cancer to an ovarian cancer therapy, and monitoring an ovarian cancer. A report may be provided to the patient of the assessment. Also provided are methods, reagents, devices and kits for the use of these biomarkers in making ovarian cancer assessments. Patients can further be treated with in accordance with the assessment of responsiveness. These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As summarized above, aspects of the subject invention include compositions, methods, systems and kits that find use in providing an ovarian cancer assessment, e.g. diagnosing, prognosing, monitoring, and/or treating ovarian cancer in a subject. In describing the subject invention, compositions useful for providing an ovarian cancer assessment will be described first, followed by methods, systems and kits for their use.

### Ovarian Cancer Biomarkers and Biomarker Panels

In some aspects of the invention, ovarian cancer biomarkers are provided. By a “biomarker” or “marker” it is meant a molecular entity whose representation in a sample is associated with a disease phenotype. By “ovarian cancer” it is meant any cancerous growth arising from the ovary, for example, a surface epithelial-stromal tumor (adenocarcinoma, including, e.g., papillary serous cystadenocarcinoma, endometrioid tumor, serous cystadenocarcinoma, papillary, mucinous cystadenocarcinoma, clear-cell ovarian tumor, Mucinous adenocarcinoma, cystadenocarcinoma, and others), a carcinoma (e.g., sex cord-stromal tumors, other carcinomas), a germ cell tumor (e.g. teratoma, Dysgerminoma, and others), Mullerian tumor, epidermoid tumor (squamous cell carcinomas), Brenner tumor, and the like, as known in the art or as described herein. Thus, by an ovarian cancer “biomarker” or “ovarian cancer marker” it is meant a molecular entity whose representation in a sample is associated with an ovarian cancer phenotype, e.g., the presence of ovarian cancer, the stage of ovarian cancer, a prognosis associated with the ovarian cancer, the predictability of the ovarian cancer being responsive to a therapy, etc. In other words, the marker may be said to be differentially represented in a sample having an ovarian cancer phenotype.

Ovarian cancer biomarkers include proteins that are differentially represented in an ovarian cancer phenotype and their corresponding genetic sequences, i.e. mRNA, DNA, etc. By a “gene” or “recombinant gene” it is meant a nucleic acid comprising an open reading frame that encodes for the protein. The boundaries of a coding sequence are determined by a start codon at the 5′ (amino) terminus and a translation stop codon at the 3′ (carboxy) terminus. A transcription termination sequence may be located 3′ to the coding sequence. In addition, a gene may optionally include its natural promoter (i.e., the promoter with which the exons and introns of the gene are operably linked in a non-recombinant cell, i.e., a naturally occurring cell), and associated regulatory sequences, and may or may not have sequences upstream of the AUG start site, and may or may not include untranslated leader sequences, signal sequences, downstream untranslated sequences, transcriptional start and stop sequences, polyadenylation signals, translational start and stop sequences, ribosome binding sites, and the like. The term “gene product” or “expression product” are used herein to refer to the RNA transcription products (transcripts) of the gene, including mRNA; and the polypeptide translation products of such RNA transcripts, i.e. the amino acid product encoded by a gene. A gene product can be, for example, an RNA transcript of the gene, e.g. an unspliced RNA, an mRNA, a splice variant mRNA, a microRNA, a fragmented RNA, etc.; or an amino acid product encoded by the gene, including, for example, full length polypeptide, splice variants of the full length polypeptide, post-translationally modified polypeptide, and fragments of the gene product, e.g. peptides, etc. In some instances, an elevated level of marker or marker activity may be associated with the ovarian cancer phenotype. In other instances, a reduced level of marker or marker activity may be associated with the ovarian cancer phenotype.

As demonstrated in the examples of the present disclosure, the inventors have identified two proteins, Prkdc and Rad54L, that are represented at elevated levels in blood samples of subtypes of ovarian cancers, and thus, that find use as biomarkers in providing an ovarian cancer assessment, e.g. diagnosing an ovarian cancer, prognosing an ovarian cancer, determining a treatment for a subject affected with ovarian cancer, monitoring a subject with ovarian cancer, and the like. The PRKDC gene, also known as “protein kinase, DNA-activated, catalytic polypeptide”, DNA-PKcs, HYRC, p350, DNAPK, DNPK1, HYRC1, and XRCC7, encodes the catalytic subunit of the DNA-dependent protein kinase (DNA-PK). It functions with the Ku70/Ku80 heterodimer protein in DNA double strand break repair and recombination. The protein encoded is a member of the PI3/PI4-kinase family. The cDNA and protein sequences for PRKDC may be found at Genbank Accession No. NM_006904.6. The RAD54L gene, also known as “RAD54-like”, HR54, hHR54, RAD54A, and hRAD54, encodes a protein that belongs to the DEAD-like helicase superfamily, and shares similarity with Saccharomyces cerevisiae Rad54, a protein known to be involved in the homologous recombination and repair of DNA, including DNA double strand break repair. The binding of this protein to double-strand DNA induces a DNA topological change, which is thought to facilitate homologous DNA paring, and stimulate DNA recombination. The cDNA and protein sequences for RAD54L may be found at Genbank Accession No. NM_003579.3.

Because Prkdc and Rad54L are critical mediators of DNA repair, ovarian cancer patients having elevated levels of Prkdc or Rad54L protein or protein activity in, e.g., blood, will be more resistant to and less responsive to cancer therapies that are DNA damaging agents than will ovarian cancer patients having Prkdc or Rad54L levels that correlate more closely to Prkdc or Rad54L levels in blood in individuals that do not have cancer. By “DNA damaging agents” it is meant chemotherapeutic agents or radiations that damage DNA, e.g. by alkylating or methylating DNA to induce mismatches, by inhibiting topoisomerase 2, by inducing breaks in DNA, etc., e.g. as known in the art or as described below. For example, ovarian cancer patients having 1.5-fold, 2-fold, 3-fold, 4-fold, 5-fold, 6-fold, 7-fold, 8-fold, 9-fold, or 10-fold or more Prkdc or Rad54L protein in their blood than an individual that does not have cancer will be less responsive to DNA damaging agents than ovarian cancer patients having levels of Prkdc or Rad54L protein in their blood that are similar to those of unaffected individuals. By being “responsive” to a cancer therapy it is meant that administration of an effective amount of agent to the subject will decrease the rate of proliferation of ovarian cancer cells, e.g. by 70%, by 80%, by 90%, or by 100%, i.e. halting the growth of the ovarian cancer cells, and in some cases induce a regression in the size of the ovarian cancer tumor or metastasis, e.g. inducing a 10% decrease or more in tumor size, e.g., a 20% decrease, a 30% decrease, a 40% decrease, a 50% decrease, or a 60% decrease in tumor size, sometimes a 70%, 80% or 90% decrease in size, in some cases eradicating visible signs of the ovarian cancer cells from the subject and inducing remission of the ovarian cancer. By being “less responsive”, it is meant that administration of an effective amount of agent to the subject may decrease the rate of proliferation of ovarian cancer cells by 30%, by 40%, by 50%, by 60% by 70%, by 80%, by 90% or by 100%, i.e. halting the growth of the ovarian cancer cells, but typically will not induce a regression in the size of the ovarian cancer tumor or metastasis. In some cases, the ovarian cancer patient having elevated Prkdc or Rad54L protein levels will be substantially unresponsive to the DNA therapy. By being unresponsive, or insensitive, to the cancer therapy, it is meant that administration of an effective amount of agent to the subject will have substantially no effect on the proliferation of ovarian cancer cells and size of the ovarian cancer tumor. Thus, the subject ovarian cancer biomarkers can be used in methods for determining whether a DNA damaging agent can be used to reduce the growth of an ovarian cancer. The methods also find use in treating subjects with a DNA damaging agent if the subject is determined to be responsive to such an agent by the methods of the invention.

Also provided in aspects of the invention are panels of ovarian cancer biomarkers. By an “ovarian cancer biomarker panel” or “ovarian cancer marker panel’ it is meant a collection, or combination, of two or more molecular entities, e.g. two, three, four, five, or more than five entities, whose representation in a sample is associated with an ovarian cancer phenotype. In some instances, the representation (level of protein, level of protein activity, level of RNA, etc.) of the biomarkers in the panel may be considered individually to make an ovarian cancer assessment. In other instances, the representation of the biomarkers may be considered in combination, e.g. together and/or with ovarian cancer biomarkers known or discovered in the art, in making an ovarian cancer assessment. Thus, in the present instance, Prkdc may be used either alone or in combination with Rad54L or with other ovarian cancer biomarkers known in the art to make an ovarian cancer assessment. Likewise, Rad54L may be used either alone or in combination with Prkdc or with other ovarian cancer biomarkers known in the art to make an ovarian cancer assessment. Any convenient ovarian cancer biomarkers may be used in combination with Prkdc and Rad54L in the subject ovarian cancer biomarker panels, for example, gene products for aldehyde dehydrogenase 1 (ALDH1), ApoC1, ApoAII, ApoCII, β-hemoglobin, Calcyclin, Calgranulin A, Calgranulin C, claudin-3, connective tissue growth factor (CTGF), eosinophil-derived neurotoxin, fibroblast growth factor 2 (basic) (FGF2), folate receptor 1 (FOLR1), glycodelin, GPCR49, glutathione S-transferase theta 1 (GSTT1), hepsin, hepcidin, insulin-like growth factor-II, inter-α-trypsin inhibitor heavy chain H4, kallikrein-related peptidase 6 (KLK6/7), kallikrein 10, leptin, macrophage inhibitory factor, mucin-16 (CA125), osteopontin, prolactin, protease serine 8 (PRSS8), Protein C inhibitor, solute carrier family 39 (zinc transporter) member 4 (SLC39A4), small MBL-associated protein C-terminal fragment, stratum corneum chymotrytic enzyme, transferrin, transthyretin, WAP four-disulfide core domain 2 (HE4), phosphorylated Src homology 2 domain containing transforming protein 1 (Shc), phosphorylated Src homology 2 domain containing E (She), and autoantibodies specific for casein kinase 1 epsilon. Biomarkers of particular interest include those directed to determining the likelihood of responsiveness of the ovarian cancer to DNA damaging drugs, e.g. as disclosed in U.S. Pat. No. 7,470,509, the full disclosure of which is incorporated herein by reference.

### Methods

The subject ovarian cancer biomarkers find use in making an ovarian cancer assessment for a patient, or “subject”. By an “ovarian cancer assessment”, it is generally meant a prediction of a subject's susceptibility to ovarian cancer, a determination as to whether a subject is presently affected by ovarian cancer, a prognosis of a subject affected by ovarian cancer (e.g., identification of ovarian cancer states, stages of the ovarian cancer, prediction of responsiveness to a therapy and/or intervention, e.g. sensitivity or resistance a chemotherapy, radiation, or surgery, likelihood that a patient will die from the ovarian cancer, etc.), and the use of therametrics (e.g., monitoring a subject's condition to provide information as to the effect or efficacy of therapy on the ovarian cancer). Thus, for example, the subject ovarian cancer biomarkers and biomarker panels may be used to diagnose ovarian cancer, to provide a prognosis to a patient having ovarian cancer, to provide a prediction of the responsiveness of a patient with ovarian cancer to a medical therapy, to monitor a patient having ovarian cancer, to treat a patient having ovarian cancer, etc.

In practicing the subject methods, an ovarian cancer biomarker signature for a patient is obtained. By an “ovarian cancer biomarker signature” or more simply, “ovarian cancer signature”, it is meant a representation of the measured level/activity (e.g., protein level, protein activity level, RNA level, etc.) of an ovarian cancer biomarker or biomarker panel of interest. A biomarker signature typically comprises the quantitative data on the biomarker levels/activity of these one or more biomarkers of interest. Examples of biomarker signatures include collections of measured protein, protein activity, and/or RNA levels. For example, a “protein biomarker signature” comprises the quantitative data on the amount of polypeptide encoded by one or more disease biomarkers. An “activity biomarker signature” comprises the quantitative data on the amount of protein activity (e.g., enzymatic activity as determined by an assay), exhibited by one or more disease biomarkers. An “RNA biomarker signature” comprises the quantitative data on the amount of RNA transcribed by one or more disease biomarkers. As used herein, the term “biomarker signature” encompasses “protein signature” and “activity signature”, as well as “RNA signature.” Examples of biomarker signatures include biomarker profiles and biomarker scores. By a “biomarker profile” it is meant the normalized representation of one or more biomarkers of interest, i.e. a panel of biomarkers of interest, in a patient sample. By a “biomarker score” it is meant a single metric value that represents the sum of the weighted representations of one or more biomarkers of interest, more usually two or more biomarkers of interest, i.e. a panel of biomarkers of interest, in a patient sample. Biomarker profiles and scores are discussed in greater detail below.

For example, in some embodiments, the subject methods may be used to obtain an ovarian cancer signature. That is, the subject methods may be used to obtain a representation of the protein, RNA, or activity levels of one or more ovarian cancer biomarkers, e.g. Prkdc or Rad54L, that are up- or down-regulated (i.e., expressed at a higher or lower level, exhibits a higher or lower level of activity, etc.), in ovarian cancers that are non-responsive to DNA damaging therapy. In certain embodiments, the ovarian cancer signature is a protein signature, comprising the quantitative data on the amount of polypeptide encoded by the one or more ovarian cancer biomarkers. In certain embodiments, the ovarian cancer signature is an activity signature, comprising the quantitative data on the amount of protein activity (e.g., enzymatic activity as determined by an assay) exhibited by one or more ovarian cancer biomarkers. In certain embodiments, the ovarian cancer signature is a ovarian cancer RNA signature, comprising the quantitative data on the amount of RNA transcribed by one or more ovarian cancer biomarkers.

To obtain an ovarian cancer signature, the protein level, protein activity level, mRNA level, etc. of the one or more ovarian cancer biomarkers of interest is detected in a patient sample. That is, the representation of one or more ovarian cancer biomarkers, e.g. Prkdc and/or Rad54L, and in some instances other ovarian cancer biomarkers in the art, e.g. a panel of biomarkers, is determined for a patient sample. The term “sample” with respect to a patient encompasses blood and other liquid samples of biological origin, solid tissue samples such as a biopsy specimen or tissue cultures or cells derived or isolated therefrom and the progeny thereof. The definition also includes samples that have been manipulated in any way after their procurement, such as by treatment with reagents; washed; or enrichment for certain cell populations. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. The term “blood sample” encompasses a blood sample (e.g., peripheral blood sample) and any derivative thereof (e.g., fractionated blood, plasma, serum, etc.).

In performing the subject methods, the biomarker level is typically assessed in a body fluid sample (e.g., a sample of blood, e.g., whole blood, fractionated blood, plasma, serum, etc.) that is obtained from an individual. The sample that is collected may be freshly assayed or it may be stored and assayed at a later time. If the latter, the sample may be stored by any convenient means that will preserve the sample so that gene expression may be assayed at a later date. For example the sample may freshly cryopreserved, that is, cryopreserved without impregnation with fixative, e.g. at 4° C., at −20° C., at −60° C., at −80° C., or under liquid nitrogen. Alternatively, the sample may be fixed and preserved, e.g. at room temperature, at 4° C., at −20° C., at −60° C., at −80° C., or under liquid nitrogen, using any of a number of fixatives known in the art, e.g. alcohol, methanol, acetone, formalin, paraformaldehyde, etc.

The sample may be assayed as a whole sample, e.g. in crude form. Alternatively, the sample may be fractionated prior to analysis, e.g. for a blood sample, to purify leukocytes if, e.g., the biomarker to be assayed is an intracellular protein, or an RNA, to purify plasma or serum if, e.g., the biomarker is a secreted polypeptide. Further fractionation may also be performed, e.g., for a purified leukocyte sample, fractionation by e.g. panning, magnetic bead sorting, or fluorescence activated cell sorting (FACS) may be performed to enrich for particular types of cells, thereby arriving at an enriched population of that cell type for analysis; or, e.g., for a plasma or serum sample, fractionation based upon size, charge, mass, or other physical characteristic may be performed to purify particular secreted polypeptides, e.g. under denaturing or non-denaturing (“native”) conditions, depending on whether or not a non-denatured form is required for detection. One or more fractions are then assayed to measure the expression levels of the one or more genes of interest. As such, the term “blood” as used herein is inclusive of whole blood as well as any fractionated portion thereof (e.g., blood cell fractions, plasma, serum, etc.).

The representation of the one or more biomarkers of interest may be measured by any convenient method known in the art for measuring protein levels, protein activity levels, polynucleotide, i.e. mRNA, levels, etc. For example, the amount or level in the sample of Prkdc or Rad54L proteins/polypeptides may be determined. Any convenient protocol for evaluating protein levels may be employed where the level of one or more proteins in the assayed sample is determined. For antibody-based methods of protein level determination, any convenient antibody can be used that specifically binds to the intended biomarker (e.g., Prkdc, Rad54L). The terms “specifically binds” or “specific binding” as used herein refer to preferential binding to a molecule relative to other molecules or moieties in a solution or reaction mixture (e.g., an antibody specifically binds to a particular polypeptide or epitope relative to other available polypeptides or epitopes). In some embodiments, the affinity of one molecule for another molecule to which it specifically binds is characterized by a KD (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). By “Affinity” it is meant the strength of binding, increased binding affinity being correlated with a lower KD. For example, Prkdc levels may be detected using antibody MA5-15813 (Pierce), 1B9 (Abnova), LS-B6857 (LifeSpan BioSciences), CIB1 (Abgent), Y393 (Abgent), or 3H6 (MyBioSource); while Rad54L levels may be detected using LS-C9873 (LifeSpan BioSciences), LS-C110146 (LifeSpan BioSciences), Sbe62 5F4/2 (Creative BioMart), 5H3 (Creative BioMart), or 4G2 (Novus Biologics). Other antibodies can be readily identified by the ordinarily skilled artisan.

While a variety of different manners of assaying for protein levels are known in the art, one representative and convenient type of protocol for assaying protein levels is ELISA. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hrs at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hr at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatograpic column or filter with a wash solution or solvent.

Alternatively, non-ELISA based-methods for measuring the levels of one or more proteins in a sample may be employed and any convenient method may be used. Representative examples known to one of ordinary skill in the art include but are not limited to mass spectrometry, proteomic arrays, xMAP™ microsphere technology, western blotting, immunohistochemistry, flow cytometry, and detection in body fluid by electrochemical sensor. In, for example, flow cytometry methods, the quantitative level of gene products of the one or more genes of interest are detected on cells in a cell suspension by lasers. As with ELISAs and immunohistochemistry, antibodies (e.g., monoclonal antibodies) that specifically bind the polypeptides encoded by the genes of interest are used in such methods. As another example, electrochemical sensors may be employed. In such methods, a capture aptamer or an antibody that is specific for a target protein (the “analyte”) is immobilized on an electrode. A second aptamer or antibody, also specific for the target protein, is labeled with, for example, pyrroquinoline quinone glucose dehydrogenase ((PQQ)GDH). The sample of body fluid is introduced to the sensor either by submerging the electrodes in body fluid or by adding the sample fluid to a sample chamber, and the analyte allowed to interact with the labeled aptamer/antibody and the immobilized capture aptamer/antibody. Glucose is then provided to the sample, and the electric current generated by (PQQ)GDH is observed, where the amount of electric current passing through the electrochemical cell is directly related to the amount of analyte captured at the electrode.

As another example, the amount or level in the sample of one or more RNAs encoded by PRKDC or RAD54L is determined. Any convenient method for measuring mRNA levels in a sample may be used, e.g. hybridization-based methods, e.g. northern blotting and in situ hybridization (Parker & Barnes, Methods in Molecular Biology 106:247-283 (1999)), RNAse protection assays (Hod, Biotechniques 13:852-854 (1992)), and PCR-based methods (e.g. reverse transcription PCR (RT-PCR) (Weis et al., Trends in Genetics 8:263-264 (1992)).

For measuring mRNA levels, the starting material may be total RNA, i.e. unfractionated RNA, or poly A+RNA isolated from a suspension of cells, e.g. a peripheral blood sample. General methods for mRNA extraction are well known in the art and are disclosed in standard textbooks of molecular biology, including Ausubel et al., Current Protocols of Molecular Biology, John Wiley and Sons (1997). RNA isolation can also be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.) or RNA Stat-60 kit (Tel-Test).

mRNA levels may be measured by any convenient method. Examples of methods for measuring mRNA levels may be found in, e.g., the field of differential gene expression analysis. One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression signatures employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Additionally or alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g. TaqMan® RT-PCR, MassARRAY® System, BeadArray® technology, and Luminex technology; and those that rely upon hybridization of probes to filters, e.g. Northern blotting and in situ hybridization.

The resultant data provides information regarding expression and/or activity for each of the ovarian cancer biomarkers that have been measured, wherein the information is in terms of whether or not the biomarker is present (e.g. expressed and/or active) and, typically, at what level, and wherein the data may be both qualitative and quantitative.

Once the representation of the one or more biomarkers has been determined, the measurement(s) may be analyzed in any of a number of ways to obtain a biomarker signature.

For example, the representation of the one or more ovarian cancer biomarkers may be analyzed individually to develop a biomarker profile. As used herein, a “biomarker profile” is the normalized representation of one or more biomarkers in a patient sample, for example, the normalized level of serological protein concentrations in a patient sample, the normalized activity of a biomarker in the sample, etc. A profile may be generated by any of a number of methods known in the art. For example, the level of each marker may be log₂ transformed and normalized relative to the expression of a selected housekeeping gene, e.g. ABL1, GAPDH, or PGK1, or relative to the signal across a whole panel, etc. Other methods of calculating a biomarker signature will be readily known to the ordinarily skilled artisan. In certain embodiments, the biomarker profile may be a “protein biomarker profile”, or simply “protein profile”, i.e. it comprises the normalized expression level(s) of the one or more biomarkers in a patient sample as determined by measuring the amount of protein encoded by the biomarker(s). In certain embodiments, the biomarker profile may be a “protein activity biomarker profile”, or simply “protein activity profile”, i.e. it comprises the normalized activity of the one or more biomarkers in a patient sample as determined by measuring the amount of protein activity exhibited in the sample. In certain embodiments, the biomarker profile may be a “RNA biomarker profile”, or simply “RNA profile”, i.e. it comprises the normalized expression level of the one or more biomarkers in a patient sample as determined by measuring the amount of RNA transcribed from the one or more biomarkers.

As another example, the measurement of an ovarian cancer biomarker or biomarker panel may be analyzed collectively to arrive at an ovarian cancer biomarker score, and the ovarian cancer biomarker signature is therefore a single score. By “biomarker score” it is meant a single metric value that represents the sum of the weighted representations of each of the biomarkers of interest, more usually two or more biomarkers of interest, in a biomarker panel. As such, in some embodiments, the subject method comprises detecting the amount/activity of markers of an ovarian cancer biomarker panel in the sample, and calculating an ovarian cancer biomarker score based on the weighted levels of the biomarkers. In certain embodiments, the biomarker score may be a “protein biomarker score”, or simply “protein score”, i.e. it comprises the weighted expression level(s) of the one or more biomarkers, e.g. each biomarker in a panel of biomarkers, in a patient sample as determined by measuring the amount of amino acid product encoded by the biomarker(s). In certain embodiments, the biomarker score may be a “protein activity biomarker score”, or simply “protein activity score”, i.e. it comprises the weighted activity of the one or more biomarkers, e.g. each biomarker in a panel of biomarkers, in a patient sample as determined by measuring the amount of activity exhibited in the sample. In certain embodiments, the biomarker score may be a “RNA biomarker score”, or simply “RNA score”, i.e. it comprises the weighted expression level of the one or more biomarkers, e.g. each biomarker in a panel of biomarkers, in a patient sample as determined by measuring the amount of RNA transcribed from the one or more biomarkers.

An ovarian cancer biomarker score for a patient sample may be calculated by any of a number of methods and algorithms known in the art for calculating biomarker scores. For example, weighted marker levels, e.g. log₂ transformed and normalized marker levels that have been weighted by, e.g., multiplying each normalized marker level to a weighting factor, may be totaled and in some cases averaged to arrive at a single value representative of the panel of biomarkers analyzed.

In some instances, the weighting factor, or simply “weight” for each marker in a panel may be a reflection of the change in analyte level in the sample. For example, the analyte level of each biomarker may be log₂ transformed and weighted either as 1 (for those markers that are increased in level in a subgroup of ovarian cancers of interest, etc.) or −1 (for those markers that are decreased in level in a subgroup of ovarian cancers of interest, etc.), and the ratio between the sum of increased markers as compared to decreased markers determined to arrive at an ovarian cancer biomarker signature. In other instances, the weights may be reflective of the importance of each marker to the specificity, sensitivity and/or accuracy of the marker panel in making the diagnostic, prognostic, or monitoring assessment. Such weights may be determined by any convenient statistical machine learning methodology, e.g. Principle Component Analysis (PCA), linear regression, support vector machines (SVMs), and/or random forests of the dataset from which the sample was obtained may be used. In some instances, weights for each marker are defined by the dataset from which the patient sample was obtained. In other instances, weights for each marker may be defined based on a reference dataset, or “training dataset”. Methods of analysis may be readily performed by one of ordinary skill in the art by employing a computer-based system, e.g. using any hardware, software and data storage medium as is known in the art, and employing any algorithms convenient for such analysis. For example, data mining algorithms can be applied through “cloud computing”, smartphone based or client-server based platforms, and the like.

Thus, in some instances, an ovarian cancer biomarker signature may be expressed as a series of values that are each reflective of the level of a different biomarker (e.g., as a biomarker profile, i.e. the normalized expression values for multiple biomarkers), while in other instances, the ovarian cancer biomarker signature may be expressed as a single value (e.g., an ovarian cancer biomarker score).

In some cases, an ovarian cancer clinical score can be integrated into an ovarian cancer biomarker signature (and/or an ovarian cancer biomarker score) such that an ovarian cancer biomarker signature (or ovarian cancer biomarker score) represents ovarian cancer biomarker data combined with ovarian cancer clinical data. Details on clinical assessments that may be and clinical scores that may be used in these embodiments are well known in the art and are described in greater detail below.

As mentioned above, in certain embodiments the expression, e.g. polypeptide level, of only one marker, e.g. Prkdc, Rad54L, is evaluated to produce a biomarker signature. In yet other embodiments, the levels of two or more biomarkers, e.g, Prkdc and/or Rad54L and one or more ovarian cancer biomarkers known in the art, e.g. as described herein, i.e. a panel of markers, e.g., 3, 4, 5, or 6 or more markers, e.g. 7, 8, 9, 10 or more markers, in some cases 12, 15, 18, or 20 or more markers, is evaluated. Accordingly, in the subject methods, the expression of at least one marker in a sample is evaluated. In certain embodiments, the evaluation that is made may be viewed as an evaluation of the proteome, as that term is employed in the art.

In some instances, the subject methods of obtaining or providing an ovarian cancer biomarker signature for a subject further comprise providing the ovarian cancer biomarker signature as a report. Thus, in some instances, the subject methods may further include a step of generating or outputting a report providing the results of an ovarian cancer biomarker evaluation in the sample, which report can be provided in the form of an electronic medium (e.g., an electronic display on a computer monitor), or in the form of a tangible medium (e.g., a report printed on paper or other tangible medium). Any form of report may be provided, e.g. as known in the art or as described in greater detail below.

The ovarian cancer signature that is so obtained may be employed to make an ovarian cancer assessment. Typically, in making the subject ovarian cancer assessment, the ovarian cancer signature is employed by comparing it to a reference or control, and using the results of that comparison (a “comparison result”) to make the ovarian cancer assessment, e.g. diagnosis, prognosis, prediction of responsiveness to treatment, etc. The terms “reference” or “control”, e.g. “reference signature” or “control signature”, “reference profile” or “control profile”, and “reference score” or “control score’ as used herein mean a standardized biomarker signature, e.g. biomarker profile or biomarker score, that may be used to interpret the ovarian cancer biomarker signature of a given patient and assign a diagnostic, prognostic, and/or responsiveness class thereto. The reference or control is typically an ovarian cancer biomarker signature that is obtained from a sample (e.g., a body fluid, e.g. blood) with a known association with a particular phenotype, for example, sensitivity to DNA damaging agents (i.e. a negative control, e.g. a sample from a healthy/unaffected individual, an individual having ovarian cancer that is sensitive to DNA damaging therapy) or resistance to DNA damaging agents (i.e. a positive control, e.g. a sample from an individual having ovarian cancer that is resistant to, i.e. nonresponsive to or refractory to, DNA damaging agents). Typically, the comparison between the ovarian cancer signature and reference will determine whether the ovarian cancer signature correlates more closely with the positive reference or the negative reference, and the correlation employed to make the assessment. By “correlates closely”, it is meant is within about 40% of the reference, e.g. 40%, 35%, or 30%, in some embodiments within 25%, 20%, or 15%, sometimes within 10%, 8%, 5%, or less.

For example, a comparison result that shows that the Prkdc- or Rad54L-based ovarian cancer biomarker signature for a patient is elevated relative to the Prkdc- or Rad54L-based ovarian cancer biomarker signature in a negative control reference (e.g. the biomarker signature of a body fluid sample from an individual that is not affected with ovarian cancer, etc.) is predictive of an insensitivity of the ovarian cancer in the patient to DNA damaging therapy. Conversely, a comparison result that reveals that a Prkdc or Rad54L based ovarian cancer signature for a patient correlates closely with the Prkdc- or Rad54L-based ovarian cancer biomarker signature of a negative control reference is predictive of sensitivity of the ovarian cancer in the patient to DNA damaging therapy.

In certain embodiments, the obtained ovarian cancer signature for a subject is compared to a single reference/control biomarker signature to obtain information regarding the phenotype. In other embodiments, the obtained biomarker signature for the subject is compared to two or more different reference/control biomarker signatures to obtain more in-depth information regarding the phenotype of the assayed tissue. For example, a biomarker profile may be compared to both a positive biomarker profile and a negative biomarker profile, or a biomarker score may be compared to both a positive biomarker score and a negative biomarker score to obtain confirmed information regarding whether the tissue has the phenotype of interest. As another example, a biomarker profile or score may be compared to multiple biomarker profiles or scores, each correlating with a particular diagnosis, prognosis or therapeutic responsiveness.

### Utility

As alluded to above, the subject biomarkers, biomarker panels, methods, reagents and kit find use in making a number of types of ovarian cancer assessments. These include, for example, in diagnosing an ovarian cancer, in classifying an ovarian cancer (e.g. stage I, stage II, stage III, or stage IV); in prognosing an ovarian cancer, in predicting the responsiveness of an ovarian cancer to a cancer therapy, in determining a therapy for an ovarian cancer patient, in monitoring an ovarian cancer, and the like. By “diagnosing” an ovarian cancer or “providing an ovarian cancer diagnosis,” it is generally meant providing an ovarian cancer determination, e.g. a determination as to whether a subject (e.g. a subject that has clinical symptoms of ovarian cancer, a subject that is asymptomatic for ovarian cancer but has risk factors associated with ovarian cancer, a subject that is asymptomatic for ovarian cancer and has no risk factors associated with ovarian cancer) is presently affected by ovarian cancer; a classification of the subject's ovarian cancer into a subtype of ovarian cancer; a determination of the severity of ovarian cancer; and the like. By “prognosing” an ovarian cancer, or “providing an ovarian cancer prognosis,” it is generally meant providing an ovarian cancer prediction, e.g. a prediction of a subject's susceptibility, or risk, of developing ovarian cancer; a prediction of the course of disease progression and/or disease outcome, e.g. expected duration of the ovarian cancer, expectation of whether the individual will die from the cancer, etc.; a prediction of a subject's responsiveness to treatment for the ovarian cancer, e.g., positive response, a negative response, no response at all; and the like. By “monitoring” an ovarian cancer, it is generally meant monitoring a subject's condition, e.g. to inform an ovarian cancer diagnosis, to inform an ovarian cancer prognosis, to provide information as to the effect or efficacy of an ovarian cancer treatment, and the like. By “treating” an ovarian cancer it is meant prescribing or providing any treatment of an ovarian cancer in a mammal, and includes: (a) preventing the ovarian cancer from occurring in a subject which may be predisposed to ovarian cancer but has not yet been diagnosed as having it; (b) inhibiting the ovarian cancer, i.e., arresting its development; or (c) relieving the ovarian cancer, i.e., causing regression of the ovarian cancer. The terms “individual,” “subject,” “host,” and “patient,” are used interchangeably herein and refer to any mammalian subject for whom diagnosis, treatment, or therapy is desired, particularly humans.

The ovarian cancer assessment may be made of any cancerous growth arising from the ovary, for example, a surface epithelial-stromal tumor (adenocarcinoma, including, e.g., papillary serous cystadenocarcinoma, endometrioid tumor, serous cystadenocarcinoma, papillary, mucinous cystadenocarcinoma, clear-cell ovarian tumor, Mucinous adenocarcinoma, cystadenocarcinoma, and others), a carcinoma (e.g., sex cord-stromal tumors, other carcinomas), a germ cell tumor (e.g. teratoma, Dysgerminoma, and others), Mullerian tumor, epidermoid tumor (squamous cell carcinomas), Brenner tumor, and the like, as known in the art or as described herein. The ovarian cancer may be of any stage, for example, stage I, stage II, stage III, or stage IV as defined by the FIGO or AJCC staging system, as known in the art and described in Table 1 below.

TABLE 1
Classification of ovarian cancers by the International Federation of
Gynaecology and Obstetrics (FIGO) staging system and the American Joint Committee on
Cancer (AJCC) staging system.

| Stage | | Pathology by FIGO | Pathology by AJCC |
|---|---|---|---|
| Stage I | IA | involves one ovary; capsule intact; no tumor on | T1a + N0 + M0 |
| | | ovarian surface; no malignant cells in ascites | |
| | | or peritoneal washings | |
| | IB | involves both ovaries; capsule intact; no tumor | T1b + N0 + M0 |
| | | on ovarian surface; negative washings | |
| | IC | tumor limited to ovaries with any of the | T1c + N0 + M0 |
| | | following: capsule ruptured, tumor on ovarian | |
| | | surface, positive washings | |
| Stage II | IIA | extension or implants onto uterus or fallopian | T2a + N0 + M0 |
| | | tube; negative washings | |
| | IIB | extension or implants onto other pelvic | T2b + N0 + M0 |
| | | structures; negative washings | |
| | IIC | pelvic extension or implants with positive | T2c + N0 + M0 |
| | | peritoneal washings | |
| Stage | IIIA | microscopic peritoneal metastases beyond | T3a + N0 + M0 |
| III | | pelvis | |
| | IIIB | macroscopic peritoneal metastases beyond | T3b + N0 + M0 |
| | | pelvis less than 2 cm in size | |
| | IIIC | peritoneal metastases beyond pelvis >2 cm or | T3c + N0 + M0 |
| | | lymph node metastases | |
| Stage | | distant metastases to the liver or outside the | Any T + Any N + M1 or |
| IV | | peritoneal cavity | Any T + N1 + M0 |

In the AJCC system, T is used to categorize the pathology of the tumor (The T1 category of ovarian cancer describes ovarian tumors that are confined to the ovaries, and which may affect one or both of them. The sub-subcategory T1a is used to stage cancer that is found in only one ovary, which has left the capsule intact and which cannot be found in the fluid taken from the pelvis. Cancer that has not affected the capsule, is confined to the inside of the ovaries and cannot be found in the fluid taken from the pelvis but has affected both ovaries is staged as T1b. T1c category describes a type of tumor that can affect one or both ovaries, and which has grown through the capsule of an ovary or it is present in the fluid taken from the pelvis. T2 is a more advanced stage of cancer. In this case, the tumor has grown in one or both ovaries and is spread to the uterus, fallopian tubes or other pelvic tissues. Stage T2a is used to describe a cancerous tumor that has spread to the uterus or the fallopian tubes (or both) but which is not present in the fluid taken from the pelvis. Stages T2b and T2c indicate cancer that metastasized to other pelvic tissues than the uterus and fallopian tubes and which cannot be seen in the fluid taken from the pelvis, respectively tumors that spread to any of the pelvic tissues (including uterus and fallopian tubes) but which can also be found in the fluid taken from the pelvis. T3 is the stage used to describe cancer that has spread to the peritoneum. This stage provides information on the size of the metastatic tumors (tumors that are located in other areas of the body, but are caused by ovarian cancer). These tumors can be very small, visible only under the microscope (T3a), visible but not larger than 2 centimeters (T3b) and bigger than 2 centimeters (T3c)); N describes the pathology of local lymph nodes (N0 indicates that the cancerous tumors have not affected the lymph nodes, and N1 indicates the involvement of lymph nodes close to the tumor); and M describes the extent, if any, of metastasis (M0 indicates that the cancer did not spread to distant organs and M1 category is used for cancer that has spread to other organs of the body). The subject biomarkers, biomarker panels, methods, reagents and kit find use in making an assessment of any ovarian cancer.

For example, the subject ovarian cancer signature finds use in predicting if an ovarian cancer will be responsive to a DNA damaging agent. As used herein, the term “agent” is defined broadly as anything that cancer cells, including tumor cells, may be exposed to in a therapeutic protocol. In the context of the present invention, such agents include, but are not limited to, chemotherapeutic agents, such as anti-metabolic agents (e.g., Ara AC, 5-FU and methotrexate), antimitotic agents (e.g., TAXOL, inblastine and vincristine), alkylating agents (e.g., nitrogen mustard, melphanlan, BCNU), Topoisomerase II inhibitors (e.g., VW-26, topotecan, mitoxantrone (DHAD)), strand-breaking agents (e.g., doxorubicin, bleomycin, procarbazine), cross-linking agents (e.g., alkylating agents such as nitrogen mustards, β-chloro-nitrosourea compounds, platinum-based compounds); radiation; ultraviolet light; and the like. By “chemotherapeutic agent” is intended to include chemical reagents which inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and are typically used to treat neoplastic diseases. By “DNA damaging agents” it is meant agents that damage DNA, e.g. by alkylating or methylating DNA, by inhibiting topoisomerase 2, by inducing single or double strand DNA breaks, etc. Examples of DNA damaging agents include chemotherapeutic and well as radiative agents, for example, alkylating agents such as Nitrogen mustards (e.g. Cyclophosphamide, Mechlorethamine (“mustine”), Uramustine (“uracil mustard”), melphanlan, Chlorambucil, Ifosfamide, and Bendamustine), β-chloro-nitrosourea compounds (e.g., Carmustine (“BCNU”), Lomustine, Semustine, and Streptozotocin, and Ethylnitrosourea (“ENU”)), Alkyl sulfonates (e.g. Busulfan), Thiotepa and Thiotepa analogues, platinum-based compounds (e.g. cisplatin, carboplatin (CBDCA), nedaplatin, oxaliplatin, satraplatin, picoplatin, phenanthriplatin, and triplatin tetranitrate), procarbazine, altretamine, dacarbazine, mitozolomide, and temozolomide; topoisomerase II inhibitors (e.g., amsacrine, etoposide, etoposide phosphate, teniposide, doxorubicin, and mitoxantrone); UV radiation and UV mimetics (e.g. N-acetoxy-2-acetylaminofluorene); Ionizing radiation; and radiomimetic agents (doxorubicin, bleomycin, and enediynes, e.g. neocarzinostatin).

For example, because Prkdc and Rad54L are critical mediators of DNA repair, ovarian cancer patients having elevated levels of Prkdc or Rad54L protein or protein activity in, e.g., blood or tumor tissue, will be more resistant and less responsive to cancer therapies that act by damaging DNA than will ovarian cancer patients having Prkdc or Rad54L levels that correlate more closely to Prkdc or Rad54L levels in individuals that do not have cancer. Thus, for example, an ovarian cancer subject can be assessed to determine a whether a DNA damaging agent can be used to reduce the growth of the ovarian cancer by obtaining a ovarian cancer signature by the methods of the present disclosure and comparing that signature to a reference signature. If the ovarian cancer signature correlates closely with an ovarian cancer reference signature of one or more patients with an ovarian cancer that is responsive to DNA damaging agents, e.g. the median across a cohort of patients with a responsive ovarian cancer, it can be predicted that the ovarian cancer of the individual of interest will be responsive to the DNA damaging agent and the DNA damaging agent can be used to reduce the growth of the ovarian cancer. In contrast, if the ovarian cancer signature is elevated relative to the ovarian cancer reference signature of the responsive ovarian cancer, e.g. elevated 1.5-fold, 2-fold, 3-fold, 4-fold, 5-fold, 6-fold, 7-fold, 8-fold, or 10-fold or more relative to the reference signature, or correlates closely with an ovarian cancer reference signature of one or more patients with an ovarian cancer that is not responsive to DNA damaging agents, e.g. the median across a cohort of patients with a non-responsive ovarian cancer, it can be predicted that the ovarian cancer of the individual of interest will not be responsive to the DNA damaging agent and the DNA damaging agent cannot be used to reduce the growth of the ovarian cancer. These predictive methods can be used to assist patients and physicians in making treatment decisions, e.g. in choosing the most appropriate treatment modalities for any particular patient.

The subject ovarian cancer signature so employed finds use in providing a prognosis to a patient having ovarian cancer. For example, a patient can be ascribed to high- or low-risk categories, or high-, medium- or low-risk categories for overall survival depending on whether their ovarian cancer biomarker signature correlates more closely with the median ovarian cancer signature across a cohort of patients having a form of ovarian cancer that is highly resistant to DNA damaging therapy or highly sensitive to DNA damaging therapy, the overall survival rates of patients with these types of ovarian cancer being known in the art or readily determined by the ordinarily skilled artisans by, e.g., Kaplan-Meier analysis of ovarian cancer individuals.

The subject ovarian cancer signature may be used on samples collected from patients in a clinical trial and the results of the test used in conjunction with patient outcomes in order to determine whether subgroups of patients are more or less likely to show a response to a new drug than the whole group or other subgroups. Further, such methods can be used to identify from clinical data the subsets of patients who can benefit from therapy. Additionally, a patient is more likely to be included in a clinical trial if the results of the test indicate a higher likelihood that the patient will be responsive to medical treatment, and a patient is less likely to be included in a clinical trial if the results of the test indicate a lower likelihood that the patient will be responsive to medical treatment.

The subject methods can be used alone or in combination with other clinical methods for patient stratification known in the art to provide a diagnosis, a prognosis, or a prediction of responsiveness to therapy. For example, clinical parameters that are known in the art for diagnosing ovarian cancer, diagnosing types of ovarian cancer, or staging ovarian cancer may be incorporated into the ordinarily skilled artisan's analysis to arrive at an ovarian cancer assessment with the subject methods.

For example, in some instances, the ovarian cancer assessment may include determining if the subject has one or more symptoms associated with ovarian cancer, e.g., bloating, abdominal or pelvic pain, difficulty eating, and/or urinary symptoms more than 12 times per month; an abdominal mass, abdominal distension, back pain, constipation, tiredness, anemia, abnormal vaginal bleeding, rectal bleeding, postmenopausal bleeding, involuntary weight loss, appetite loss, and/or a build-up of fluid (ascites) in the abdominal cavity. In some instances, making an ovarian cancer assessment includes the step of determining if the subject has one or more symptoms associated with ovarian cancer, e.g. as described above or known in the art; wherein the ovarian cancer assessment is made based on the ovarian cancer signature and the symptom determination.

As another example, in some instances, ovarian cancer assessment may include determining if the subject has one or more risk factors associated with ovarian cancer, e.g., advanced age (63 years old or older); obesity (a body mass index of 30 or more); a family history of ovarian cancer, breast cancer or colorectal cancer (a first or second degree relative with the disease); a personal history with breast cancer; reproductive history (an increase risk associated with women who have never given birth); a genetic mutation associated with ovarian cancer (e.g., in BRCA1, in BRCA2, in genes for hereditary nonpolyposis colorectal cancer); infertility; a history of endometriosis; and/or a history of use of postmenopausal estrogen replacement therapy; wherein the ovarian cancer assessment is made based on the ovarian cancer signature and the risk determination.

As another example, in some instances, the ovarian cancer assessment may include characterizing the tumor, e.g., by the aforementioned FIJO or AJCC staging system, by histochemistry or immunohistochemistry of a tumor sample, by the use of biomarkers known in the art for assessing an ovarian cancer, etc.; wherein the ovarian cancer assessment is made based on the ovarian cancer signature and the tumor characterization. In certain instances, the ovarian cancer signature used to make the ovarian cancer assessment includes the representation of these other biomarkers, for example, the ovarian cancer signature on which the ovarian cancer assessment is made is an ovarian cancer score that is reflective of the subject Prkdc and/or Rad54L levels in the subject's blood as well as levels of other known biomarkers. Any convenient ovarian cancer biomarker(s), for example as known in the art or described herein, may be used in combination with Prkdc and Rad54L to obtain an ovarian cancer signature and provide an ovarian cancer assessment. Non-limiting examples include the gene products for aldehyde dehydrogenase 1 (ALDH1), ApoC1, ApoAII, ApoCII, β-hemoglobin, Calcyclin, Calgranulin A, Calgranulin C, claudin-3, connective tissue growth factor (CTGF), eosinophil-derived neurotoxin, fibroblast growth factor 2 (basic) (FGF2), folate receptor 1 (FOLR1), glycodelin, GPCR49, glutathione S-transferase theta 1 (GSTT1), hepsin, hepcidin, insulin-like growth factor-II, inter-α-trypsin inhibitor heavy chain H4, kallikrein-related peptidase 6 (KLK6/7), kallikrein 10, leptin, macrophage inhibitory factor, mucin-16 (CA125), osteopontin, prolactin, protease serine 8 (PRSS8), Protein C inhibitor, solute carrier family 39 (zinc transporter) member 4 (SLC39A4), small MBL-associated protein C-terminal fragment, stratum corneum chymotrytic enzyme, transferrin, transthyretin, WAP four-disulfide core domain 2 (HE4), phosphorylated Src homology 2 domain containing transforming protein 1 (Shc), phosphorylated Src homology 2 domain containing E (She), and autoantibodies specific for casein kinase 1 epsilon. Biomarkers of particular interest include those directed to determining the likelihood of responsiveness of the ovarian cancer to DNA damaging drugs, e.g. as disclosed in U.S. Pat. No. 7,470,509, the full disclosure of which is incorporated herein by reference.

### Reports

In some embodiments, providing an ovarian cancer signature or providing an ovarian cancer assessment, e.g., a diagnosis of ovarian cancer, a prognosis for a patient with ovarian cancer, a prediction of responsiveness of a patient with ovarian cancer to a cancer therapy, includes generating a written report that includes that ovarian cancer signature and/or the ovarian cancer assessment e.g., a “diagnosis assessment”, a “prognosis assessment”, a suggestion of possible treatment regimens (a “treatment assessment”) and the like. Thus, the subject methods may further include a step of generating or outputting a report providing the results of an analysis of an ovarian cancer biomarker or biomarker panel, a diagnosis assessment, a prognosis assessment, or a treatment assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a diagnosis assessment, a prognosis assessment, a treatment assessment, a monitoring assessment, etc. and its results. A subject report can be completely or partially electronically generated. A subject report includes at least an ovarian cancer assessment, e.g., a diagnosis as to whether a subject has a high likelihood of having an ovarian cancer that is resistant to DNA damaging therapy; or a prognosis assessment, e.g. a prediction of the responsiveness of a patient to a DNA damaging therapy; and/or a suggested course of treatment to be followed. A subject report can further include one or more of: 1) information regarding the testing facility; 2) service provider information; 3) subject data; 4) sample data; 5) an assessment report, which can include various information including: a) test data, where test data can include i) the biomarker levels of one or more ovarian cancer biomarkers; and/or ii) the biomarker signatures for one or more ovarian cancer biomarkers.

The report may include information about the testing facility, which information is relevant to the hospital, clinic, or laboratory in which sample gathering and/or data generation was conducted. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including subject medical history as well as administrative subject data (that is, data that are not essential to the diagnosis, prognosis, or treatment assessment) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the susceptibility prediction and, if different from the ordering physician, the name of a staff physician who is responsible for the subject's care (e.g., primary care physician).

The report may include a sample data section, which may provide information about the biological sample analyzed, such as the source of biological sample obtained from the subject (e.g. blood, e.g., whole blood, fractionated blood, plasma, serum, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, flash drive, etc.

It will be readily appreciated that the report can include all or some of the elements above, with the proviso that the report generally includes at least the elements sufficient to provide the analysis requested by the user (e.g., a diagnosis, a prognosis, or a prediction of responsiveness to a therapy).

### Reagents, Devices and Kits

Also provided are reagents, devices and kits thereof for practicing one or more of the above-described methods. The subject reagents, devices and kits thereof may vary greatly. Reagents and devices of interest include those mentioned above with respect to the methods of assaying gene expression levels, where such reagents may include protein or RNA purification reagents, reagents for measuring protein activity, antibodies to the subject ovarian cancer biomarker proteins (e.g., immobilized on a substrate, e.g., in the form of a dipstick, i.e., lateral flow assay device), nucleic acid primers specific for ovarian cancer biomarker RNAs, arrays of nucleic acid probes, signal producing system reagents, etc., depending on the particular detection protocol to be performed. For example, reagents may include antibodies that are specific for Prkdc or Rad54L, arrays that comprise probes that are specific for Prkdc or Rad54L; or other reagents that may be used to detect the level of Prkdc or Rad54L in blood.

The subject kits may also comprise one or more biomarker signature references, e.g. a reference for an ovarian cancer signature, for use in employing the biomarker signature obtained from a patient sample. For example, the reference may be a sample of a known phenotype, e.g. an unaffected individual, or an affected individual, e.g. from a particular risk group that can be assayed alongside the patient sample, or the reference may be a report of disease diagnosis, disease prognosis, or responsiveness to therapy that is known to correlate with one or more of the subject ovarian cancer biomarker signatures.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Example 1

### Materials and Methods

Data Collection, Pre-Processing, and Normalization.

Gene expression data for 7 human ovarian cancer studies were downloaded from the NCBI GEO (Table 2). Data sets were filtered to include only normal and high grade serous ovarian cancer samples. All data sets were separately normalized using gcRMA.

Meta-Analysis.

Two meta-analyses approaches were applied to the normalized data. The first approach combines effect sizes from each data set into a meta-effect size to estimate the amount of change in expression across all data sets. For each gene in each data set, an effect size was computed using Hedges' adjusted g. If multiple probes mapped to a gene, the effect size for each gene was summarized using the fixed effect inverse-variance model. Next, study-specific effect sizes were combined to obtain the pooled effect size and its standard error using the random effects inverse-variance technique. The z-statistic was computed as a ratio of the pooled effect size to its standard error for each gene, and compared the result to a standard normal distribution to obtain a nominal p-value. P-values were corrected for multiple hypotheses testing using FDR.

We also applied a second non-parametric meta-analysis that combines p-values from individual experiments to identify genes with a significant effect in all data sets. A t-statistic was calculated for each gene in each study. After computing one-tail p-values for each gene, they were corrected for multiple hypotheses using FDR. Fisher's sum of logs methods was applied. Briefly, this method sums the logarithm of FDR-corrected p-values across all data sets for each gene, and compares the sum against a chi-square distribution with 2 k degrees of freedom, where k is the number of data sets used in the analysis.

To control for the influence of single large experiments on the meta-analysis results, leave-one-out meta-analysis was performed. One data set at a time was excluded and both meta-analysis methods were applied to the remaining data sets. Only genes that were identified as significantly over-expressed with a large effect size in all 7 leave-one-out analyses were considered for further analysis.

TABLE 2
Studies used for meta-analysis. To select these studies, I searched GEO for all
ovarian cancer experiments on human samples that included both normal and ovarian
cancer samples. Only those studies using Affy arrays were included. The 8^{th} study
identified meeting these criteria was used as a validation cohort.

| GEO ID | Cases | Controls | Description | Location |
|---|---|---|---|---|
| GSE26712 | 185 | 10 | A Gene Signature Predicting for Survival in | NCI |
| | | | Suboptimally Debulked Patients with Ovarian | |
| | | | Cancer, 185 primary ovarian tumors and 10 | |
| | | | normal ovarian surface epithelium | |
| GSE25427 | 12 | 10 | Gene expression profiles of primary cultured | Duke |
| | | | ovarian cells in the presence and absence of a | |
| | | | DNA methyltransferase inhibitor, 12 serous | |
| | | | primary cultures and 2 pooled normal ovarian | |
| | | | surface epithelium | |
| GSE6008 | 41 | 4 | Human ovarian tumors and normal ovaries | U of |
| | | | | Michigan |
| GSE19352 | 17 | 4 | Activation of phosphatidylcholine-cycle enzymes | Instituto |
| | | | in human epithelial ovarian cancer cells, 17 EOC | Superiore di |
| | | | frozen surgical specimens, 3 pooled and 1 | Sanita |
| | | | separate OSE | (Rome) |
| GSE18520 | 53 | 10 | Whole-genome oligonucleotide expression | MD |
| | | | analysis of papillary serous ovarian | Anderson |
| | | | adenocarcinomas, We identified 53 advanced | |
| | | | stage, high-grade primary tumor specimens and | |
| | | | 10 normal ovarian surface epithelium (OSE) | |
| | | | brushings | |
| GSE14001 | 10 | 3 | PAX2: A Potential Biomarker for Low Malignant | MD |
| | | | Potential Ovarian Tumors and Low-Grade | Anderson |
| | | | Serous Ovarian Carcinomas, RNA from 3 | |
| | | | normal human ovarian surface epithelia (HOSE) | |
| | | | and from 10 high-grade serous ovarian | |
| | | | carcinoma samples | |
| GSE10971 | 13 | 24 | Gene expression data from non-malignant | U of Toronto |
| | | | fallopian tube epithelium and high grade serous | |
| | | | carcinoma. laser capture microdissected non- | |
| | | | malignant distal FTE from 12 known BRCA1/2- | |
| | | | mutation carriers (FTEb) and 12 control women | |
| | | | (FTEn) during the luteal and follicular phase, as | |
| | | | well as 13 high grade tubal and ovarian SerCa | |
| Total | 331 | 65 | | |

ELISAs.

Serum samples were purchased from BioServe. ELISA kits were purchased from commercial vendors specified in Table 3. Manufacturer protocols were followed for each assay. Briefly, all reagents and samples were brought to room temperature and the samples were centrifuged before beginning the assay. 100 ul of sample serum or kit standard was placed in each well (standards were run in duplicate). Wells were incubated for 2 hours at 37° C. Liquid was removed and 100 ul of biotin-antibody was added to each well. Wells were incubated for 1 hour at 37° C. and then washed 3×. 100 ul of HRP-avidin was added to each well, incubated for 1 hour at 37° C., and washed 6×. 90 ul of TMB substrate was added to each well and incubated for 30 minutes in the dark at 37° C. 50 ul of Stop Solution was added to each well and the optical density at 450 nm was read with a microtiter plate reader within 15 minutes.

TABLE 3
Kits used for ELISAs

| | | | Serum |
|---|---|---|---|
| | | | Dilution |
| Protein | Vendor | Catalog Number | for Assay |
| CA-125 | Abcam | ab108653 | 1:1 |
| Osteopontin (OPN) | Abcam | ab100618 | 1:10 |
| IGF2 (ILGF2) | Creative Diagnostics | DEIA731 | 1:100 |
| Leptin | Abcam | ab100581 | 1:100 |
| RAD54L | MyBioSource | MBS906598 | 1:10 |
| PRKDC | MyBioSource | MBS932608 | 1:10 |

### Results

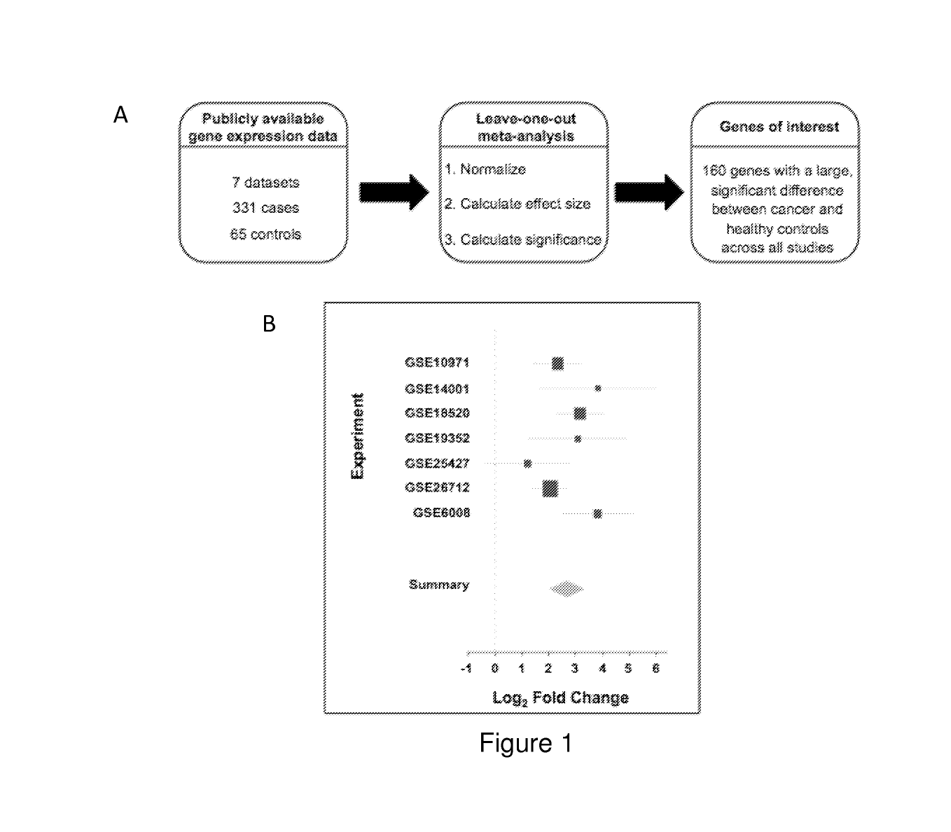

7 datasets (Table 2) containing gene expression data of both high grade serous ovarian cancer and controls were downloaded from The Gene Expression Omnibus. Gene expression data were normalized, the summary statistic (also called the effect size) was calculated, and the statistical significance was determined. Genes were then ranked based on the summary statistic and the statistical significance. Based on the filters of log fold change>0.75, p-value<0.001, FDR<0.001, 160 genes were identified that are statistically significantly overexpressed across all 7 studies reviewed (FIG. 1).

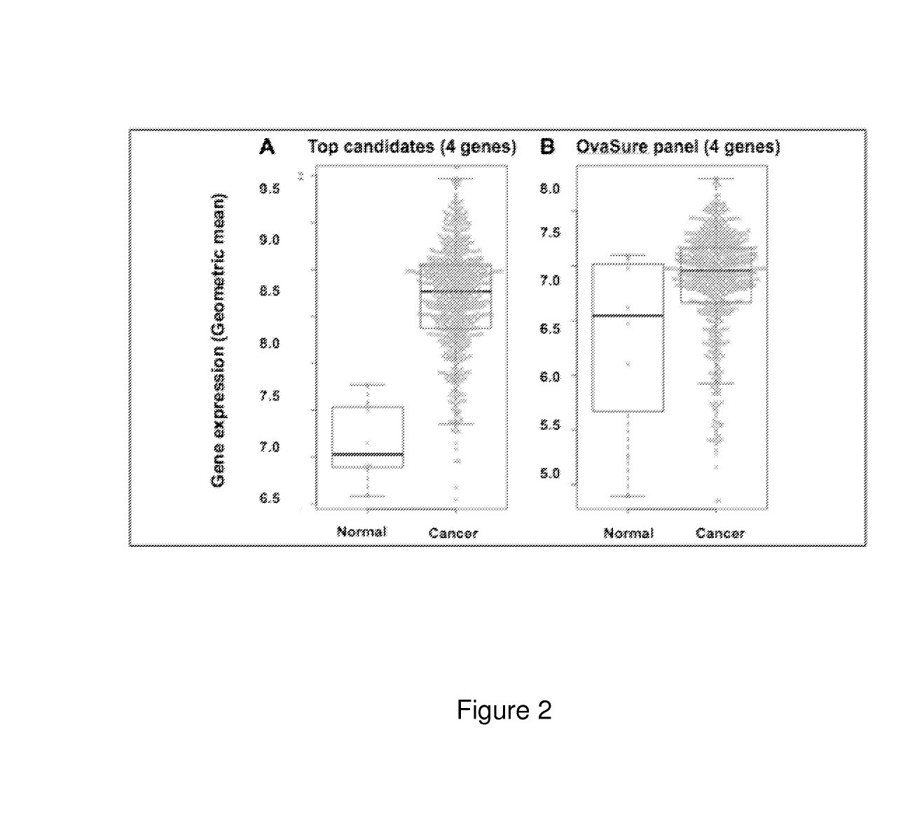

To confirm the association between the candidate biomarkers identified by these methods and ovarian cancer, the ability of a representative panel of 4 candidate biomarkers to distinguish ovarian cancer cases from normal cases was compared to that of OvaSure, a biomarker panel of 4 overexpressed proteins (FIG. 2). The analysis was performed using data from The Cancer Genome Atlas (TCGA), which has expression data from 591 ovarian cancer cases, of which 46 cases are early stage (stage I or II) and the rest are late stage. The 4 genes identified by our methods as having the highest effect size were used as the representative panel. To arrive at a score, the geometric mean of the expression values of the 4 genes was calculated from the TCGA microarray data (the 4 values were multiplied together and then the 4^{th} root taken). We observed a better separation between normal individuals and cancer patients using the new panel of candidate biomarkers (median score for normal individuals=7; for cancer patients=8.5) than OvaSure (median score for normal individuals=6.5; for cancer patients=7).

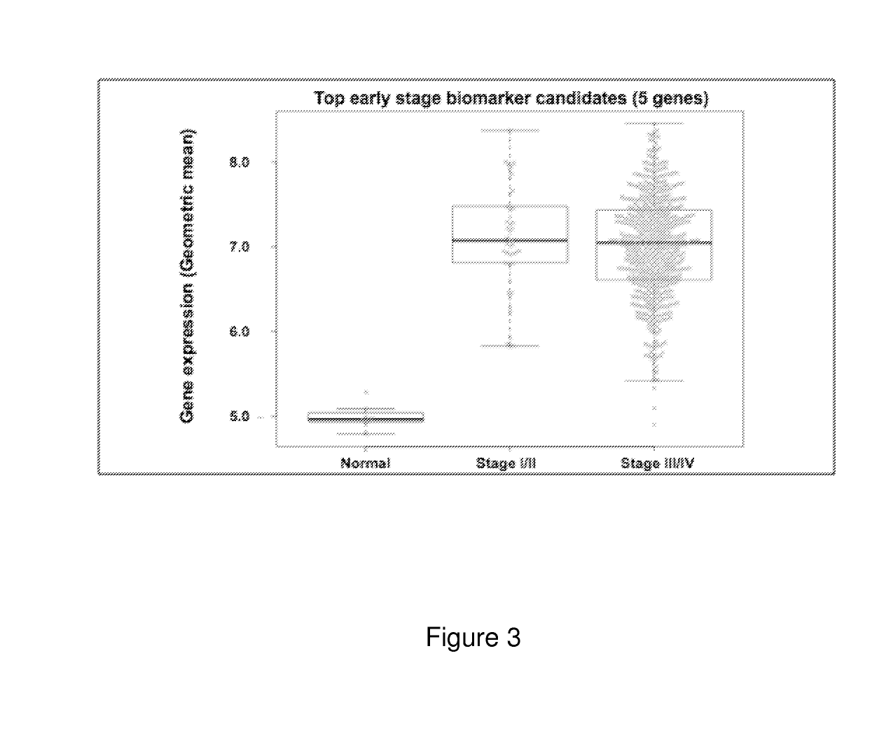

Using a t-test, the 160 candidate biomarkers were ranked based on how well they distinguish between normal and early stage cases (i.e. stage I or stage II). Only those 80 genes having a p-value (adjusted for multiple hypothesis testing) of <0.05 were considered further as potential early ovarian cancer biomarkers. Those 80 early detection candidates were then ranked based on how well they correlated with survival, under the assumption that a gene that is correlated with survival is more likely to have a functional role in cancer and therefore be more likely to be a robust biomarker. A panel of the top 5 genes from this ranked list performed well at separating normal from early and late stage cases (FIG. 3), indicating that this panel could be used in the detection of both early and late stage ovarian cancers.

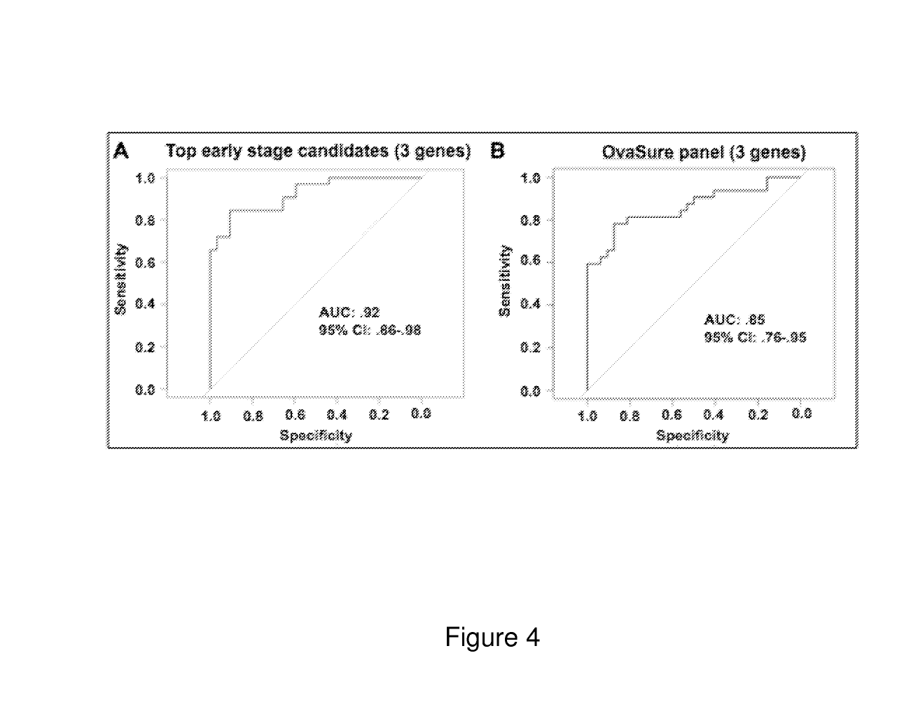

Early detection candidates were validated using a separate independent dataset (GSE4122) consisting of 32 ovarian serous adenocarcinoma cases and 32 normal or benign controls. A panel of the top 3 candidates achieved a higher AUC than the 3 OvaSure genes (FIG. 4), indicating that our early detection candidates outperform OvaSure.

The list of candidate biomarkers from meta-analysis were filtered against proteome databases, and 2 proteins (PRKDC and RAD45L) were selected for a pilot validation study in the plasma of 12 pre-treatment ovarian cancer patients and 12 age-, gender-, and ethnicity-matched controls. 4 of the 6 biomarkers in the discontinued OvaSure panel (CA125, OPN, LEP, ILGF2) were tested as positive controls. Serum concentrations of the individual proteins that comprise the OvaSure panel were similar to those reported previously in cases and controls (Vinistin et al., 2008). PRKDC serum concentrations distinguish cases and controls at a magnitude similar to the OvaSure proteins and is statistically significant (p=0.0055). RAD45L serum concentrations are very low in the healthy controls. Within the cancer cases, there are two very distinct subpopulations with low or elevated serum levels, in contrast to the gradient observed within cancer samples for the other proteins measured.

PRKDC and RAD45L are known to be important in the repair of DNA double-strand breaks. PRKDC is a key protein for non-homologous end joining (NHEJ) downstream of BRCA1 and ATM proteins. It has also been shown to play a direct role in resistance to the chemotherapy agent Cisplatin and inhibitors are being tested in early phase clinical trials. Levels of PRKDC, measured by immunohistochemistry (IHC), are used to predict treatment response in prostate cancer, and IHC levels of this protein have been shown to correlated with metastasis and survival in ovarian cancer. We have shown for the first time that elevated PRKDC can also be detected in the serum by ELISA. Therefore, in addition to being a promising candidate for inclusion in a serum biomarker panel for early-detection of ovarian cancer, it may also be used to predict and/or monitor treatment response. RAD54L directly binds to RAD51 and is a key protein for homologous repair (HR) downstream of BRCA1 and ATR proteins. Inhibitors of RAD51 are in early phase clinical trials and reduced RAD51 may increase sensitivity to PARP inhibitors. Since we show that only a subset of high grade ovarian cancer patients have elevated serum levels of RAD54L, a blood test can be used to determine which patients are good candidates for RAD51 and PARP inhibitors as part of their treatment plan. Patients can further be treated with a RAD51 or PARP inhibitor if they are determined to be good candidates.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of providing an ovarian cancer signature for a subject, the method comprising:
evaluating the level of one or more ovarian cancer biomarkers in a blood sample from a subject; and
calculating the ovarian cancer signature based on the level of the one or more ovarian cancer biomarkers in the blood sample.

2. The method according to claim 1, wherein the one or more ovarian cancer biomarkers is selected from Prkdc protein and/or Rad54L protein.

3. The method according to claim 1, wherein the subject has an ovarian cancer.

4. The method according to claim 1, wherein the evaluating comprises the use of an antibody.

5. The method according to claim 1, further comprising preparing a report of the ovarian cancer signature.

6. An ovarian cancer biomarker panel, the panel comprising Prkdc and/or Rad54L.

7. A method for making an ovarian cancer assessment for a subject, comprising:
obtaining an ovarian cancer signature for a subject based on a blood sample from the subject;
comparing the ovarian cancer signature for the subject to an ovarian cancer signature for a reference; and
making an ovarian cancer assessment based on the comparison.

8. The method according to claim 7, wherein the ovarian cancer signature is obtained by:
evaluating the level in the blood sample of one or more ovarian cancer biomarkers selected from Prkdc protein and/or Rad54L protein; and
calculating the ovarian cancer signature based on the level of the one or more ovarian cancer biomarkers in the blood sample.

9. The method according to claim 8, wherein the evaluating comprises detecting the one or more ovarian cancer biomarkers with an antibody.

10. The method according to claim 7, wherein the assessment is a prediction of the responsiveness of the subject to a DNA damaging agent.

11. The method according to claim 10, wherein the DNA damaging agent is radiation.

12. The method according to claim 10, wherein the DNA damaging agent is a platinum-based compound.

13. The method according to claim 12, wherein the platinum-based compound is selected from cisplatin, carboplatin, nedaplatin, oxaliplatin, satraplatin, picoplatin, phenanthriplatin, and triplatin tetranitrate.

14. The method according to claim 7, wherein the assessment is classifying the ovarian cancer.

15. The method according to claim 14, wherein the classifying comprises classifying the ovarian cancer into a class selected from sensitivity to a DNA damaging agent, and resistance to a DNA damaging agent.

16. The method of claim 15, further comprising treating the patient in accordance with the classification.

17. A method of monitoring an ovarian cancer subject, the method comprising:
obtaining a first ovarian cancer signature for the subject based on a blood sample from the subject;
administering a DNA damaging agent in an amount effective to treat the cancer,
obtaining a second ovarian cancer signature for the subject based on a blood sample from the subject,
comparing the second ovarian cancer signature to the first ovarian cancer signature; and
monitoring the subject based on the comparison.

18. The method according to claim 17, wherein the obtaining of the first and second ovarian cancer signatures comprises evaluating the level of one or more ovarian cancer biomarkers selected from Prkdc protein and/or Rad54L protein in a blood sample from a subject; and
calculating the ovarian cancer signature based on the level of the one or more ovarian cancer biomarkers in the blood sample.

19. The method according to claim 16, wherein an increase in Prkdc protein and/or Rad54L protein level indicates that the patient is becoming less sensitive to the DNA damaging therapy.

20. A kit for obtaining an ovarian cancer signature for a subject, the kit comprising
a binding element that is specific for Prkdc or a binding element that is specific for Rad54L; and
an ovarian cancer reference.
